SEQUENCE LISTING (7 sequences)

SEQ ID NO: 1 - DNA, 720 bp, synthetic construct
  misc_feature 1..720: note = Chemically Synthesized
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 2 - DNA, 709 bp, synthetic construct
  misc_feature 1..709: note = Chemically Synthesized
  source 1..709: mol_type = other DNA
cagaatacatggacggtgttcagagatgccattacccaaactggaacgggacctgccttcttgaccatcaaggaatggatcgaaagagggactaccaaaagcatggaggcagcgaacatcatgagcaatcttccgacgactgttcgcacgccgactgacagctacatcagatccttcttcgaactgttacaaaatcctaaagtgagcaacgagcaattcctcaacaccgccgccaccttatcgttctgcgagatgatccacaatgcccaagtgaacaagaggagcattcacaataattatccagtccacacgttcggtcgtcttacgtcgaaacacgacaacagcctctacgacgagtacataccattcctggaacgcgagctgaggaaagcccatcaagaaaaggacagcccaaggatccagacctacatcatggctctgggaatgatcggggaacccaagattctgtccgtgttcgagccatatctggaagggaaacagcagatgaccgtgttccagaggacgttgatggtcggctctttgggcaaattaaccgaaactaatccgaaattggcacgctcggttctgtacaagatttatctgaacacgatggagagccacgaagtccgttgcaccgcggtattccttctgatgacgaccaatccaccgttgagcatgttgcaacgaatggcggaattcaccaagttgg

SEQ ID NO: 3 - DNA, 708 bp, synthetic construct
  misc_feature 1..708: note = Chemically Synthesized
  source 1..708: mol_type = other DNA
acgacaacacatccctcgcccttccaaagaacgagggaccgttcaggttaaacgtcgaaactgctaaaactaacgaggaaatgtgggaactgatcgataccgagaaattgaccgatcgtctcccctacccctggaccatggacaacgagagatacgtgaaggtagacatgtacatgaatttggagggggagcagaaagatcctgtgatattctccacgtcgttcgacagcaaggtgatgacacggcccgacacggattcagagaactggacaccgaaaatgatggcggtcgagccgacggacaagcaggcgaacagcaagactcgtaggcaagaaatgatgagggaagcgggaaggggtatcgaatcggcgaaatcgtacgtggtggacgtacgagtgcacgttcctggcgagtcggagagcgagactgttctcaccctggcctggtcggagagcaacgtggagagtaaggggcgattgctcggtttctggcgcgtcgaaatgccgaggagcaacgccgattacgaggtttgcatcggctctcagatcatggtttcgccggaaactcttctgtcctacgacgagaagatggaccagaagccgaagatggatttcaacgtggatatccgttacgggaagaactgcggcaagggcgagaggatcgatatgaacggtaaacttaggcagagccctaggcttaaggaattgg

SEQ ID NO: 4 - DNA, 994 bp, synthetic construct
  misc_feature 1..994: note = Chemically Synthesized
  source 1..994: mol_type = other DNA
gaagtttgcaagatgctgtatgtggtgtgcctggattagatgggtttgattcgatatcgtggaatactagtgctggttttcctttgtctccattaaagccacctggaacatcaggtaagcgatggttgtttgatattgagctacaagactcgggatgttatctcctgcgtggaatgcgtcccgaacttgagattcaattatcaacgacacagttaatgaggaaaaagggaataaaacctcacactatattcacggattgtttgaaagatacttgtttgcctgttgaaaaatgtagaatacctggtaagactagaatatttagtataagtccggtacagtttaccataccgtttcgacagtattacctagactttatggcatcctatcgagctgcacgacttaatgctgagcatggtattggtattgatgttaacagcttagagtggacaaatttggcaacaaggttgtctaagtatggcactcacatcgtgacaggagactataagaattttggtcctgggttagattccgatgttgcagcttcagcgtttgaaattattatcgactgggtattacattacaccgaagaagataataaagacgaaatgaagcgagtaatgtggaccatggcgcaagagatcttagcgcctagtcatctatatcgcgatttagtgtaccgagtaccttgcggaattccatcaggttctccaataacggacatattgaatacaatttcaaattgtttgttaattaggttagcttggttaggtattactgatttgcctttgtccgagttctctcaaaatgttgttcttgtctgttatggcgacgatcttatcatgaatgttagcgataacatgattgataagtttaacgccgtgacgataggaaaattcttttcacaatataagatggaatttacggatcaggataaatcaggaaatactgtaaagtggcggacgttacagactgctactttcttaaaacatgggtt

SEQ ID NO: 5 - DNA, 500 bp, synthetic construct
  misc_feature 1..500: note = Chemically Synthesized
  source 1..500: mol_type = other DNA
cctgagtgtaagagcattattaaatttatagcgtcacataatgagcatatacgtgctcagaatgatggagtgttagtaactggcgaccatactcagctattggctttcgagaataataataaaactccaataagtattaacgctgatggtttgtatgaggttatacttcaaggagtatatacttatccataccacggcgatggtgtttgtggttcgatattgttatctcggaatttacaacggccaattataggtatccatgttgctggtactgagggattgcatggttttggagtcgctgaaccacttgtacatgaaatgttcaccggtaaagcgatcgagagtgaaagagagccgtatgatcgtgtgtatgaacttccgttgcgtgaattagatgaatctgatattggtttagataccgatttatatccgattggtagagtggatgcaaagctagctcatgctcaaagcccttctactgggattaaaaagacgcttat

SEQ ID NO: 6 - DNA, 1000 bp, synthetic construct
  misc_feature 1..1000: note = Chemically Synthesized
  source 1..1000: mol_type = other DNA
atacagggctaagacaggttatgcaccatattatgctggagtgtggcatagctttaataatagtaattctcttgtttttaggtggggatctgcttctgaccaaattgctcagtggccgacaatttcagtaccaagaggtgagttagctttcttacgaattaaggatggaaagcaagctgctgtaggaactcaaccttggcgtacgatggttgtttggccttctggtcacggctataatattggtatacccacgtataatgctgaacgagctcgccagcttgcacaacacttatatggtggtggatcattaactgatgagaaggccaaacaattatttgttcctgctaatcaacaaggacctggtaaggtaagtaatggaaatccggtatgggaagtcatgcgtgcaccattgtcaacacagcgtgcgcatatgcaagattttgaatttattgaggctattccagaaggagaggagtctcgtaatactacagtcttggatacgaccactactttacagtcgagtgggtttggtcgcgccttctttggagaagcttttaatgatcttaaaacgttaatgcggcgatatcaattatatggtcaattattattgtccgttactacggataaggatattgatcattgtatgtttaccttcccttgtttaccacaagggttagcgttagacattggttctgctggctctccacatgaaatctttaatagatgtcgtgatggtattataccattaattgcatctggatatagattttatagaggagatttgcgttataagattgtttttccaagtaatgttaatagcaacatttgggtacaacatcgaccggatcgtagactggaaggatggtccgcggctaagattgtaaattgtgatgctgtgtctactggtcaaggagtgtataatcatggttatgctagtcacattcaaatcacgcgtgtaaataatgttatagaattggaagttccattttataatgctact

SEQ ID NO: 7 - DNA, 5268 bp, synthetic construct
  misc_feature 1..5268: note = Chemically Synthesized
  source 1..5268: mol_type = other DNA
atggattttcagcacaattggcaagtgggcaacgagtacacttacctcgtgcgttcgagaactctgacaagcctcggtgatttgtccgatgtacatacgggaatcctaatcaaggctctactgacggttcaggccaaggattccaacgttcttgcggctaaagtgtggaatggccaatatgcgcgcgtgcaacagagtatgcctgatgggtgggaaaccgaaatttcagatcagatgttagaactccgtgatttaccgattagtggcaaacccttccagatccgcatgaaacatgggctcatccgggatttaatcgtcgaccgggacgttcccacctgggaagttaatattttgaaaagcattgtggggcagctccaggttgacactcaaggcgagaatgccgtcaaagttaattcggtgcaagtccccacggatgatgaaccctacgcgagctttaaagccatggaagatagcgttggcggcaaatgtgaagttctgtacgatatcgctcccctgtccgattttgttatccatcgcagcccggaactggtcccgatgcctacattaaagggggatgggcgccacatggaagttatcaagatcaagaattttgataactgtgaccaacgtattaattatcattttggcatgaccgataatagtcggctggagcccggaacaaacaaaaacggcaaatttttctcccggagctcaacctcacgcattgttatcagcgaatctttaaagcattttaccatccagtcgtcagtaaccacgtcgaaaatgatggtgagtccgcgcctgtatgaccggcaaaacggattggtgctgtctcgcatgaatctgacgttggcaaaaatggaaaagacatcgaagcctctaccaatggtggacaatcccgaatctacgggcaacttagtctacatttacaataacccatttagcgatgttgaagagcgtcgcgtgtctaagaccgctatgaattcgaatcagattgtttccgataactcgctgtcaagcagtgaagagaagctgaagcaggacatcctgaacctgcggaccgatatttcaagcagttcatcaagcatctcaagtagtgaggaaaacgatttttggcaaccgaaaccgacgctggaggatgccccccaaaacagtctcctcccgaattttgtgggctataagggcaaacacatcggtaagtccggcaaggtggacgtaattaacgctgccaaagagctgatttttcaaattgctaatgaattggaggatgccagtaatatcccggtgcacgctacgctggaaaaatttatgattttatgtaatctgatgcgcaccatgaatcggaaacagattagtgaactagaaagcaacatgcaaatctcgccgaatgagttgaaaccgaacgataaatctcaggtgattaagcagaatacctggaccgtctttagagatgccatcacccagacgggcaccggccccgctttcctgacgatcaaagaatggatcgaacgcggtaccacgaagtccatggaggccgcaaatatcatgagcaagctccccaaaaccgtgcgtacaccgaccgattcatatatccggagcttcttcgagctgcttcagaatcctaaggtgagcaatgaacagttcctcaataccgccgccaccttatcgttctgcgaaatgattcacaacgctcaggtcaataaacgatcgatccacaataactacccggttcatacctttggtcgtctcacttccaaacacgacaacagtctatatgatgaatatattccctttctggagcgcgagctgcgaaaggcccaccaggaaaaagacagcccgcgcatccaaacgtacatcatggcacttggcatgatcggagagcccaaaattctaagcgtctttgagccgtacctggagggtaagcagcagatgactgtgttccaacgtaccttgatggttggtagcctgggtaagttgaccgagacgaacccgaagctcgcccgctccgtcctgtacaaaatctatttaaataccatggaaagtcatgaggtgcgctgtactgcagtttttctgctcatgaaaactaatccgccgctgtcaatgctgcagcgaatggcagagttcaccaagctggacactaaccgacaggtcaatagcgccgtcaaatccacgattcaatccttaatgaagctgaagagtccagaatggaaggatctagccaagaaggcccgaagtgttaatcacttgctcacacatcatgagtacgattacgaattatcgcgcggttatatcgatgaaaagattttggagaaccaaaatatcatcactcacatgattttaaactatgtcggtagcgaagattcagttatcccccgcattctgtatctcacatggtactcgtcaaacggcgatatcaaggttccttcaaccaaggtcttggccatgatctcgtcagtgaagtcattcatggagctctcgttgcgatctgtgaaggaccgcgaaaccattatctcggccgccgagaagatcgccgaagaactgaagattgtccccgaagaattggtccccttggagggtaatctaatgattaacaacaaatatgccctcaagttcttccctttcgacaagcatatcttagataaactgcctacccttatttctaattacatcgaggcggtcaaagaaggaaagttcatgaatgtgaacatgttagatacgtacgagtccgtacacagttttccaaccgaaacgggcttacctttcgtttatactttcaacgtcattaagctgaccaaaaccagtggtacggtgcaagctcagatcaacccagattttgccttcatcgttaacagtaacttacgcctcactttttctaagaacgtccaagggcgcgttggatttgttacccccttcgagcaccgccatttcattagtggcattgatagcaatttgcacgtttacgccccactgaagatctctctggatgtgaatacgcctaaagggaacatgcaatggaaaatctggccgatgaagggagaggaaaaaagccgcttgtttcattactcggtggtgccgtttgtgtcaaaccacgacatcctgaatttgcgtcccctttcaatggagaaaggcacgcgacccatgattccggatgataatacctctctggcactgcccaaaaatgaaggcccgtttcgcctgaacgttgagacagccaagaccaacgaagagatgtgggagttgatcgacacagaaaaactcacggaccgcttgccctacccctggaccatggataacgaacggtacgtcaaggttgatatgtacatgaacttggagggcgaacagaaagatcccgttatcttctccacctccttcgactccaaagttatgactcgtccggataccgattctgagaattggacacccaaaatgatggcagtcgagcccactgataagcaagccaactccaaaacccgccgccaagaaatgatgcgagaagcgggccgcggtattgaatccgcaaaaagctatgtggtggatgtccgggtccacgtccccggcgaaagcgaaagcgagacggtcctcaccctcgcttggagtgagagcaatgtggaatctaaaggccgcttattgggattttggcgcgtcgaaatgccgcgctctaatgcagattacgaagtctgtattggttcccagattatggtttcacccgaaaccctgctcagctacgacgaaaaaatggaccagaagccaaaaatggacttcaatgtggacattcgttatggcaaaaattgcgggaagggcgaacggatcgacatgaatggtaagctccgacaaagtcctcgtctgaaggagttggtcggcgcaacgtctattatcaaagattgtgtcgaagatatgaaacgaggcaataaaatcttgcgcacctgccagaaggccgttgtgctctccatgcttcttgatgaagtcgatatttcaatggaagtcccgagtgacgccctgatcgccctctactcccagggattgttctcactctcggagattgacaatctcgatgtgtccctggatgttagcaacccgaagaacgcaggcaagaagaaaatcgatgtccgcgccaagttgaacgagtaccttgataaggcggacgttattgtcaatacgccgattatggatgcacactttaaggatgtgaagttgtcggactttgggttttccacagaagatattttagataccgcggatgaggacttgctaatcaacaacgtcttttacgaagatgagacgtcgtgcatgctggacaaaactcgcgcgcaaacgtttgatgggaaagattatccgctccgcctgggcccttgttggcatgccgttatgacgacttatccccgcatcaatccggataatcataatgagaaattgcatattccgaaagataagtcggtcagtgtgctgagccgtgaaaatgaagctggccaaaaagaagtcaaggttctgttggggagtgacaaaatcaaatttgtgcccgggaccaccagtcagccggaggttttcgtcaacggcgaaaagattgttgttagccgcaacaaagcgtatcagaaagttgaagaaaacgaaatcattttcgaaatatacaaaatgggcgaccgttttatcggcttgaccagtgacaaatttgatgtgagtctggccttggatggggaacgcgtcatgctgaaggcatctgaagattaccgctacagcgtccgtggcctgtgcggaaattttgatcacgacagtaccaacgattttgtgggtcccaaaaattgcttgttccgaaaaccggaacactttgttgctagttacgcgttgatctccaatcagtgtgaaggcgacagtctaaacgtcgcaaagagtctgcaagatcatgattgcatccgccaagagcgcacgcaacagcgaaatgtaattagcgactccgagtccgggcgcttggatacggaaatgagcacctggggttaccaccacaatgtgaataagcactgcacaattcatcgtactcaagtgaaagagactgacgataaaatctgctttacgatgcgcccagtggtcagttgcgcgagtggctgcacggctgtcgaaacaaagtctaaaccgtataaatttcactgcatggagaaaaacgaggcagcgatgaaattaaaaaaacgcattgaaaaaggtgccaacccggatttgtcgcagaaacccgtcagcacaaccgaggaacttactgtcccgtttgtttgtaaggcatag

(19) United States
(12) Patent Application Publication
(10) Pub. No.: US 2023/0272395 A1
(43) Pub. Date: Aug. 31, 2023

(54) ENGINEERED MICROALGAE FEED TO IMPROVE HONEY BEE PATHOGEN RESISTANCE AND NUTRITION

(71) Applicant: The United States of America, as represented by The Secretary of Agriculture, Washington, DC (US)

(72) Inventors: VINCENT A. RICIGLIANO, BATON ROUGE, LA (US); MICHAEL SIMONE-FINSTROM, BATON ROUGE, LA (US); JAMES G. THOMSON, EL CERRITO, CA (US)

(21) Appl. No.: 18/175,394

(22) Filed: Feb. 27, 2023

Related U.S. Application Data
(60) Provisional application No. 63/314,495, filed on Feb. 28, 2022.

(51) Int. Cl. C12N 15/113; A01N 63/60; A01P 1/00; A61K 31/713; A23K 10/16; A23K 20/153; A23K 50/90

(52) U.S. Cl. CPC C12N 15/113; A01N 63/60; A01P 1/00; A61K 31/713; A23K 10/16; A23K 20/153; A23K 50/90; C12N 2310/14; C12N 2320/32

(57) ABSTRACT

The present disclosure provides recombinant microalgae that can deliver both pathogen protection and essential nutrition to honey bees. The microalgae can contain genetic modifications that result in the expression of RNA-interference (RNAi) inducing elements that target honey bee pathogens and endogenous honey bee genes.

### CROSS-REFERENCE

The present application claims priority to U.S. Provisional Patent Application Ser. No. 63/314,495 filed Feb. 28, 2022, the contents of which are expressly incorporated herein by reference.

### BACKGROUND OF THE INVENTION

### Field of Invention

The present disclosure provides recombinant prokaryotic microalgae that can deliver both pathogen protection and essential nutrition to honey bees. The microalgae can contain genetic modifications that result in the expression of RNA-interference (RNAi) inducing elements that target honey bee pathogens and endogenous honey bee genes.

### Background

Honey bee pollination services are essential to agricultural crop production, which represents an estimated economic value of ˜$15 billion annually in the United States alone. However, managed honey bee colonies are experiencing annual losses that are on average twice as high as historical records, threatening the beekeeping industry and food security in the United States (Kulhanek et al., J. Apiculture Res., (2017) 56:328-40). These losses are attributed to several interacting stressors, but pathogens, parasites, and poor nutrition are the primary factors that are driving colony mortality (Dolezal et al., Roy. Soc. Open Sci., (2019) 6:181803; Dolezal et al., Curr. Opin. Insect Sci., (2018) 26:114-119; Degrandi-Hoffman & Chen, Curr. Opin. Insect Sci., (2015) 10:170-76). The technology described herein can address these major honey bee threats using a novel and sustainable approach.

Currently, there are few methods for treating bee diseases, and even fewer methods that can be practically applied to bee colonies on a large scale. Hives are routinely fed artificial “pollen substitute” diets to compensate for inadequate natural food sources in the environment (Mattila & Otis, Apidologie, (2006), 37:533-46; DeGrandi-Hoffman et al., J. Insect Physiol., (2010) 56:1184-91; De Jong et al., J. Apiculture Res., (2015) 48:34-37). Nutritious feed additives that can combat diseases could take advantage of this ubiquitous management practice as a delivery system into beehives. Our research indicates the potential of prokaryotic microalgae as engineerable bee feed ingredients that can deliver edible therapeutics and essential nutrition.

### SUMMARY OF THE INVENTION

The present disclosure provides a recombinant prokaryotic microalga transformed with a heterologous nucleic acid encoding a double-stranded ribonucleic acid (dsRNA) that targets a gene expressed by a bee or a gene expressed by a bee pathogen resulting in knockdown or silencing of the dsRNA gene target within the bee. In some embodiments, the bee is a honey bee, such as a domesticated bee of the species Apis mellifera, Apis cerana, or another domesticated bee such as Bombus impatiens. In some embodiments, the prokaryotic microalga is Synechococcus elongatus, Arthrospira platensis, and Arthrospira maxima. In specific embodiments, the microalga is Synechococcus elongatus. In some embodiments, the bee pathogen targeted is a honey bee pathogen, such as a virus pathogen (e.g., Deformed Wing Virus (DWV)). In some embodiments, the prokaryotic microalgae comprises a lipid that is an essential nutrient for the bee. Further disclosed herein are bee feeds containing recombinant prokaryotic microalgae described in this paragraph.

The present disclosure also provides a method of inducing RNA interference (RNAi) in a bee or bee pathogen by 1) contacting an artificial bee feed with the prokaryotic microalgae described in the previous paragraph with a bee; and 2) allowing the bee to ingest the prokaryotic microalgae, thereby ingesting an effective amount of dsRNA produced by said prokaryotic microalgae to induce RNAi. In some embodiments, the bee is a honey bee, such as a domesticated bee of the species Apis mellifera, Apis cerana, or Bombus impatiens. In some embodiments, the microalga is Synechococcus elongatus, Arthrospira platensis, or Arthrospira maxima.

Further provided herein is a double-stranded RNA (dsRNA) having the sequence of SEQ ID NO: 4.

### SEQUENCE LISTING

The instant application contains a Sequence Listing XML required by 37 C.F.R. § 1.831(a) which has been submitted in XML file format via the USPTO patent electronic filing system and is hereby incorporated by reference in its entirety. The XML file was created on Feb. 26, 2023, is named Sequence Listing_001921.xml, and has 19.1 KB.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1 provides a schematic overview of an exemplary dsRNA expression vector.

FIG. 2 provides pictorial overview of microalgae culture scale up, diet formulation, and delivery to honey bees.

FIG. 3A and FIG. 3B provides graphical depiction of data demonstrating engineered algae strains can silence endogenous honey bee genes. FIG. 3A: Feeding two algae strains expressing dsRNA against the essential honey bee gene vitellogenin were sufficient to reduce transcript abundance 5.1- to 8-fold relative to the non-specific dsRNA control (YFP). This result demonstrates that dsRNA-producing algae can elicit an RNAi response in bees. FIG. 3B: Silencing vitellogenin led to significantly reduced lifespan in bees fed the vitellogenin dsRNA strains since this gene is implicated in longevity.

FIG. 4 provides graphical depiction of data showing engineered microalgae strains fed to honey bees reduce viral titers of Deformed Wing Virus (DWV). Bees were fed diets of either wild type (WT) or engineered algae expressing fragments DWV1-3 for 4 days. Bees were then experimentally infected with DWV and viral titers were measured using qPCR and DWV plasmid standards for absolute quantification.

FIG. 5 provides graphical depiction of data showing honey bee consumption of various microalgae diets.

FIG. 6 provides graphical depiction of data showing body weight increasing in honey bees consuming various microalgae diets, indicating their relative nutritional value.

FIG. 7 provides graphical depiction of data showing the effects of microalgae diets on mRNA expression levels of vitellogenin, a biomarker of diet quality in honey bees.

FIG. 8 provides graphical depiction of quantitation of dsRNA production in engineered strains of the prokaryotic microalga Synechococcus elongatus. Seven independent transformants from each of two different dsRNA constructs (top and bottom panels) were screened by quantitative PCR using plasmid standard curves. Total RNA extracts were either tested directly or treated with RNaseIII, which specifically degrades dsRNA. Fractions of the RNA extracts that were treated with RNaseIII yielded minimal to no amplification, indicating that amplification of untreated extracts is attributable to dsRNA production by the engineered strains.

FIG. 9 provides graphical representation of stably transformed microalgae strains fed to honey bees reducing viral titers of Deformed Wing Virus (DWV). Bees were fed diets formulated with either a vector control strain (no dsRNA expression), a YFP-expressing strain (non-specific dsRNA control) or strains expressing fragments DWV1 and DWV2 for 4 days. Bees were then injected with DWV and viral titers were measured 72 hours post injection using qPCR and DWV plasmid standards for absolute quantification.

FIG. 10 provides graphical representation of engineered microalgae diets improving survival of bees experimentally infected with Deformed Wing Virus (DWV). Bees were fed diets formulated with either a vector control strain (no dsRNA expression), a YFP-expressing strain (non-specific dsRNA control), or two microalgae strains expressing fragments DWV1 and DWV2 for 4 days. Bees were then injected with DWV and post injection survival was recorded daily. In agreement with reduced viral titers, experimentally infected bees that fed on the DWV1 and DWV2 strains had significantly improved survival outcomes.

FIG. 11 provides graphical representation of engineered microalgae diets activating the honey bee RNA interference (RNAi) immune response. Bees were fed diets comprised of either a vector control strain (no dsRNA expression) or two algae strains expressing fragments DWV1 and DWV2 for 4 days. Bees were sampled on day 5 and analyzed for dicer mRNA expression via qPCR. Dicer induces activation of the RNA-induced silencing complex, which is essential for RNAi. Bees fed the DWV1 and DWV2 strains had significantly higher dicer expression relative to bees fed the vector control diet.

FIG. 12 provides graphical representation of engineered microalgae diets expressing fragments DWV1 and DWV2 conferring viral cross-resistance to bees experimentally infected with Chronic Bee Paralysis Virus (CBPV). Bees were fed diets formulated with either a vector control strain (no dsRNA expression), a YFP-expressing strain (non-specific dsRNA control), or two microalgae strains expressing fragments DWV1 and DWV2 for 4 days. Bees were topically infected with CBPV and post infection survival was recorded daily. Bees that fed on the DWV1 and DWV2 strains had significantly improved survival outcomes, indicating that these strains can confer resistance to CBPV.

### DETAILED DESCRIPTION OF THE INVENTION

Herein, we disclose technology to produce engineered prokaryotic microalgae strains for the delivery of RNA-interference (RNAi) therapeutics that can confer resistance to bee pathogens. While application of RNAi treatment in honey bees has been demonstrated, so far no approaches meet the criteria of sustainability, cost effective scalability, and ease of application required for their deployment on a large scale (Yu et al., Insect Sci., (2013) 20:4-14). The technology described herein employs microalgae as photosynthetic dsRNA production platforms that are sustainably grown in large volumes with minimal inputs. This approach has potential for stacking multiple pathogen targets as well as the manipulation of nutritional traits (i.e; ratios of amino acids, fatty acids, and sterols) in engineered feed. Microalgae-based feeds and their modification to act as edible treatments have significant promise for addressing the major bee stressors of pathogens and malnutrition simultaneously in the practical delivery systems disclosed herein. The present disclosure provides engineered strains of the microalga Synechococcus elongatus UTEX 2973 and demonstrates their efficacy as RNAi therapeutics for bees. The novel approaches described herein could be used in other microalgae such as Arthrospira, a genus that is currently grown on an industrial scale.

Our research indicates that tested microalgae reproduce the nutritional efficacy and functional properties of natural pollen based on a variety of bee physiology and health measures. Thus, microalgae-based feeds can have significant potential to improve the health, sustainability, and productivity of managed honey bees.

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of fungal genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, N.Y., 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The terms “about” and “approximately” are defined as plus or minus ten percent of a recited value. For example, about 1.0 g means 0.9 g to 1.1 g and all values within that range, whether specifically stated or not.

The term “a nucleic acid consisting essentially of”, and grammatical variations thereof, means nucleic acids that differ from a reference nucleic acid sequence by 20 or fewer nucleic acid residues and also perform the function of the reference nucleic acid sequence. Such variants include sequences that are shorter or longer than the reference nucleic acid sequence, have different residues at particular positions, or a combination thereof.

For the purpose of the invention, the “complement of a nucleotide sequence X” is the nucleotide sequence which would be capable of forming a double-stranded DNA or RNA molecule with the represented nucleotide sequence, and which can be derived from the represented nucleotide sequence by replacing the nucleotides by their complementary nucleotide according to Chargaff's rules (A<>T; G<>C; A<>U) and reading in the 5′ to 3′ direction, i.e., in opposite direction of the represented nucleotide sequence.

As used herein, “dsRNA” refers to double-stranded RNA that comprises a sense and an antisense portion of a selected target gene (or sequences with high sequence identity thereto so that gene silencing can occur), as well as any smaller double-stranded RNAs formed therefrom by RNAse or dicer activity. Such dsRNA can include portions of single-stranded RNA, but contains at least 19 nucleotides double-stranded RNA. A dsRNA can comprise a hairpin RNA which contains a loop or spacer sequence between the sense and antisense sequences of the gene targeted. Such hairpin RNA spacer region can be any known in the art or later discovered. Particular dsRNA species can be referred to by their sequence, whether presented as a DNA or RNA sequence.

Included in this definition are “siRNAs” or small interfering (double-stranded) RNA molecules of 16-30 bp, 19-28 bp, or 21-26 bp, e.g., such as the RNA forms that can be created by RNAseIII or dicer activity from longer dsRNA. siRNAs as used herein include any double-stranded RNA of 19 to 26, or 21 to 24 base pairs that can interfere with gene expression when present in a cell wherein such gene is expressed. siRNA can be synthetically made, expressed and secreted directly from a transformed cell or can be generated from a longer dsRNA by enzymatic activity. These siRNAs can be blunt-ended or can have overlapping ends. Also, modified microRNAs comprising a portion of a target gene and its complementary sequence are included herein as dsRNAs.

The term “control”, and grammatical variants thereof, is utilized in several contexts herein. Within experiments, a “control” is a means by which experimental variables are tested to eliminate as a cause of observed results. With regards to diseases, the term “control” is intended to refer to all processes wherein there may be a slowing, interrupting, arresting, or stopping of the progression of the diseases and conditions described herein (e.g., viral infection), but does not necessarily indicate a total elimination of all disease and condition symptoms, and is intended to include prophylactic treatment. With regards to organisms (e.g., insects, bacteria, viruses, etc.), the term “control” as used herein refers to any means for preventing infection or infestation, reducing the population of already infected areas, or elimination of population(s) whose “control” is desired. Indeed, “controlling” as used herein refers to any indicia of success in prevention, elimination, reduction, repulsion, or amelioration of a target population or a problem caused by the target population (e.g., microbe, etc).

The term “effective amount” of a composition provided herein refers to the amount of the composition capable of performing the specified function for which an effective amount is expressed. The exact amount required can vary from composition to composition and from function to function, depending on recognized variables such as the compositions and processes involved. An effective amount can be delivered in one or more applications. Thus, it is not possible to specify an exact amount, however, an appropriate “effective amount” can be determined by the skilled artisan via routine experimentation.

The term “honey bee” or “honeybee” refers to any domesticated bee belonging to the genus Apis, such as Apis mellifera and Apis cerana. The term “bee” can refer to a honeybee, as well as any other domesticated bee, such as Bombus impatiens.

As used herein, “preventing” a disease refers to inhibiting the full development of a disease.

For the purpose of this invention, the “sequence identity” of two related nucleotide or amino acid sequences, expressed as a percentage, refers to the number of positions in the two optimally aligned sequences which have identical residues (x100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman & Wunsch, J. Mol. Biol., (1970) 48:3, 443-53). A computer-assisted sequence alignment can be conveniently performed using a standard software program such as GAP which is part of the Wisconsin Package Version 10.1 (Genetics Computer Group, Madison, Wis., USA) using the default scoring matrix with a gap creation penalty of 50 and a gap extension penalty of 3.

The term “prokaryotic microalgae” and grammatical variants thereof refers to blue-green algae (prokaryotic) organisms. The term includes the exemplary organisms Synechococcus elongatus, Arthrospira platensis, and Arthrospira maxima.

A “vector” is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An “expression vector” is a vector that contains the necessary regulatory sequences to allow transcription and translation of inserted sequences (e.g., dsRNAs).

A first nucleic acid sequence is “operably linked” with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences are generally contiguous and, where appropriate, can be in the same reading frame.

As used herein, the term “promoter” refers to a polynucleotide that in its native state is located upstream or 5′ to a translational start codon of an open reading frame (or protein-coding region) and that is involved in recognition and binding of RNA polymerase and other proteins (trans-acting transcription factors) to initiate transcription. The term can include promoters produced through the manipulation of known promoters to produce artificial, chimeric, or hybrid promoters. Such promoters can also combine cis-elements from one or more promoters, for example, by adding a heterologous regulatory element to an active promoter with its own partial or complete regulatory elements. The term “cis-element” refers to a cis-acting transcriptional regulatory element that confers an aspect of the overall control of gene expression. A cis-element may function to bind transcription factors, trans-acting protein factors that regulate transcription. Some cis-elements bind more than one transcription factor, and transcription factors may interact with different affinities with more than one cis-element.

Double-Stranded RNA and RNA Interference

As disclosed herein, transgenic microalgae expressing one or more dsRNAs are utilized to deliver the dsRNA to a target (e.g., honey bee). Such dsRNAs can target pathogens, including viruses and parasites, as well as host genes. Induction of RNAi of targeted pathogen genes will preferably disrupt the life cycle of the pathogen, decrease pathogenicity, or otherwise decrease the ability of the pathogen to cause disease. Induction of RNAi silencing of endogenous honey bee genes will preferably enhance honey bee survival and health.

Preferably, the dsRNAs to be used in this invention target at least one insect or pathogen gene of interest and comprise at least 19 consecutive nucleotides occurring in identical sequence or with high sequence identity in the one or more target genes. In preferred embodiments of this invention, such dsRNAs do not silence off-target host genes, or the genes of other non-target animals, such as humans, beneficial insects, pest predators, or wildlife such as reptiles, amphibians, birds, or mammals. Levels of identity between sequences of interest can be analyzed in available databases, e.g., by a BLAST search (see also www.ncbi.nlm.nih.gov/BLAST) or by hybridization with existing DNA libraries of representative non-target organisms.

As used herein, nucleotide sequences of RNA molecules can be identified by reference to DNA nucleotide sequences of the sequence listing. However, the person skilled in the art will understand whether RNA or DNA is meant depending on the context. Furthermore, the nucleotide sequence is identical between the types of polynucleotides except that the T-base is replaced by uracil (U) in RNA molecules.

In some embodiments, the length of the first (e.g., sense) and second (e.g., antisense) nucleotide sequences of the dsRNA molecules of the invention can vary from about 10 nucleotides (nt) up to a length equaling the length in nucleotides of the transcript of the target gene. The first and second sequences can be referred to as first and second strands. Additionally, it is understood that either the first or second sequence can be the sense or antisense strand. The length of the first or second nucleotide sequence of the dsRNA of the invention can be at least 15 nt, or at least about 20 nt, or at least about 50 nt, or at least about 100 nt, or at least about 150 nt, or at least about 200 nt, or at least about 400 nt, or at least about 500 nt.

It will be appreciated that the longer the total length of the first (sense) nucleotide sequence in the dsRNA of the invention is, the less stringent the requirements for sequence identity between the total sense nucleotide sequence and the corresponding sequence in the target gene becomes. The total first nucleotide sequence can have a sequence identity of at least about 75% with the corresponding target sequence, but higher sequence identity can also be used such as at least about 80%, at least about 85%, at least about 90%, at least about 95%, about 100%. The first nucleotide sequence can also be identical to the corresponding part of the target gene. However, it is preferred that the first nucleotide sequence includes a sequence of 19 or 20, or about 19 or about 20 consecutive nucleotides, or even of about 50 consecutive nucleotides, or about consecutive 100 nucleotides, or about 150 consecutive nucleotides with only one mismatch, preferably with 100% sequence identity, to the corresponding part of the target gene. For calculating the sequence identity and designing the corresponding first nucleotide sequence, the number of gaps should be minimized, particularly for the shorter sense sequences.

The length of the second (antisense) nucleotide sequence in the dsRNA of the invention is largely determined by the length of the first (sense) nucleotide sequence and may correspond to the length of the latter sequence. The nucleotide sequence of the antisense region is largely determined by the nucleotide sequence of the sense region and may be identical to the complement of the nucleotide sequence of the sense region. Particularly with longer antisense regions, it is however possible to use antisense sequences with lower sequence identity to the complement of the sense nucleotide sequence, such as at least about 75% sequence identity, or least about 80%, or at least about 85%, more particularly with at least about 90% sequence identity, or at least about 95% sequence to the complement of the sense nucleotide sequence. Nevertheless, it is preferred that the antisense nucleotide sequence includes a sequence of about 19 or about 20 consecutive nucleotides, although longer stretches of consecutive nucleotides such as about 50 nucleotides, or about 100 nucleotides, or about 150 nucleotides with no more than one mismatch, preferably with 100% sequence identity, to the complement of a corresponding part of the sense nucleotide sequence can also be used.

In one embodiment of the invention, a dsRNA molecule may further comprise one or more regions having at least 94%, at least 95%, at least 96%, at least 97%, at least 98%, or at least 99%, sequence identity to regions of at least 19 consecutive nucleotides from the sense nucleotide sequence of the target gene, different from the at least 19 consecutive nucleotides as defined in the first region, and one or more regions having at least 94%, at least 95%, at least 96%, at least 97%, at least 98%, or at least 99%, sequence identity to at least 19 consecutive nucleotides from the complement of the sense nucleotide sequence of the target gene, different from the at least 19 consecutive nucleotides as defined in the second region, wherein these additional regions can base-pair amongst themselves.

Microalgae

Various prokaryotic microalgae species can be utilized to practice the disclosure provided herein. Some non-limiting examples include Synechococcus elongatus, Arthrospira platensis, and Arthrospira maxima. Other candidate microalgal species with potential use as feed additives include those that meet honey bee essential amino acid requirements for arginine, histidine, isoleucine, leucine, lysine, methionine, phenylalanine, threonine, tryptophan, and valine (e.g; Arthrospira spp.). Microalgae with high lipid contents, especially those high in linoleic acid and alpha-linolenic acid, two fatty acids that are considered essential for bees, are also promising candidates for bee feed.

It will be apparent to one skilled in the art that different molecular methodologies (e.g., transformation protocols), sequence modifications (e.g., codon optimization), and vector components (e.g., origin of replication or selectable markers) can be adjusted to utilize the present disclosure in different organisms. For prokaryotic microalgae, dsRNA expression is driven by a target sequence that is flanked by two parts: (1) a part containing an upstream terminator followed by a strong, constitutive prokaryotic promoter without a ribosome binding site and (2) a part containing an inverted strong, constitutive prokaryotic promoter without a ribosome binding site and with a flanking terminator sequence (an exemplary system is provided in the Examples for Synechococcus).

Abundant floral resources are required for honey bee brood production, immune function, and overwinter survival. Pollen is the sole source of proteins, lipids, and micronutrients. Bee keepers feed “pollen substitute” diets to offset periods of inadequate pollen forage and to increase colony strength prior to pollination services. Several commercially available and beekeeper-formulated diets are used in beekeeping operations throughout the United States and 87% of US beekeepers claim to feed supplemental nutrition. The protein content of pollen is important for the colony. In particular, the amino acids arginine, histidine, isoleucine, leucine, lysine, methionine, phenylalanine, threonine, tryptophan, and valine are considered essential for honey bees. Pollen also contains lipids that are essential to bee physiology. Different feed ingredients have been used as a partial or full replacement for natural pollen. Artificial diets typically incorporate protein-rich ingredients such as soy, pea, yeast, casein, and egg. Some formulations include a fraction of bee-collected pollen, which can increase consumption and brood rearing. Development of alternative feed formulations that mimic the chemical composition of pollen are needed to offset bee nutritional deficiencies related to habitat loss and global change. Sustainable feed additives that do not compete with human food production and that can be engineered as functional ingredients (i.e, therapeutics), such as microalgae, are good candidates to address this crucial need of modern beekeeping. Microalgae are highly nutrient dense and can be further developed for added nutritional value by engineering strains for increased lipid content or recombinant expression of honey bee nutritional storage proteins (i.e., Vitellogenin).

The embodiments illustratively disclosed herein suitably may be practiced in the absence of any element [e.g., method (or process) steps or composition components)] which is not specifically disclosed herein. Thus, the specification includes disclosure by silence. Written support for a negative limitation may also be found through the absence of the excluded element in the specification, known as disclosure by silence.

Having generally described this invention, the same will be better understood by reference to certain specific examples, which are included herein to further illustrate the invention and are not intended to limit the scope of the invention as defined by the claims.

### EXAMPLES

### Example 1

Blue-Green, Prokaryotic Alga Synechococcus Elongatus as a dsRNA Production and Delivery Mechanism for Honey Bee RNA Therapeutics

Culture conditions for S. elongatus: The microalga S. elongatus UTEX 2973 (acquired from the University of Texas at Austin culture collection (utex.org)) was maintained on 1.5% (w/v) agar plates containing BG11 (Blue-Green) medium. Liquid cultures were grown in Erlenmeyer flasks (100 ml) containing BG11 medium shaken at 100 rpm. Larger liquid culture volumes were grown in 250 ml followed by 3000 ml bottles aerated with filter-sterilized atmospheric air. Cultures were grown at 40° C. with ˜300 umol photons m⁻²S⁻¹ under continuous light in a Percival I-36VL incubator with warm white LED lighting.

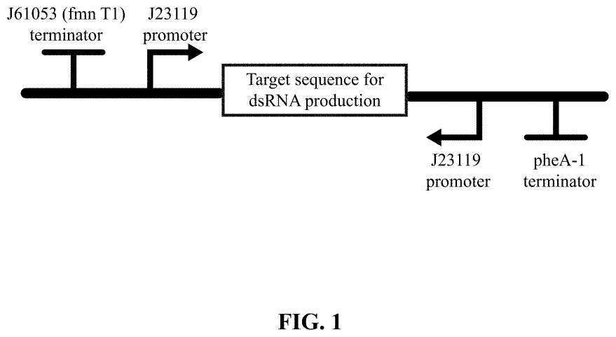

dsRNA expression vector construction: Replicative vector pPMQAK1-T carrying an RSF1010 replicative origin (Vasudevan et al., Plant Physiol., (2019) 20:4-14) was used to construct all vectors. All other parts were designed in silico using sequences obtained from GenBank and synthesized by Integrated DNA Technologies. We designed dsRNA expression parts to be compatible with vector pPMQAK1-T and golden gate assembly methods. We designed two parts: (1) a promoter part with an upstream terminator J60153 (from FMN-T1) followed by the strong synthetic J23119 promoter (Huang et al., Nucl. Acids Res., (2010) 38:2577-93) without a ribosome binding site and (2) a part containing an inverted J23119 promoter without a ribosome binding site and with a flanking terminator sequence (from pheA-1) (FIG. 1). We combined vector pPMQAK1-T plus the aforementioned parts with compatible target DNA parts to express dsRNA of the target sequence. Target sequences were designed based on GenBank accessions for honey bee vitellogenin (Vg, NM_001011578) and Deformed Wing Virus (DWV, MG831200.1) and synthesized de novo by Integrated DNA technologies Golden Gate assembly reactions were performed as previously described (Werner et al., Bioengineered, (2012) 3:38-43) except enzyme BsaI-HFv2 (New England Biolabs) was used to increase assembly efficiency. Assemblies were transformed into E. coli DH5 alpha cells and sequences were verified with sanger sequencing. We also generated integrative constructs for stable transformation of dsRNA expression constructs that rely on recombination at neutral sites within the S. elongatus UTEX 2973 genome. These sites can be disrupted with no, or minimal, impact on growth (Pinto et al, DNA Res., (2015) 22:425-37. Vector pPMQAK1-T (Vasudevan et al., Plant Physiol., (2019) 20:4-14) was combined with the aforementioned dsRNA expression parts and pathogen target sequences, a spectinomycin resistance cassette, and flanking sequences for homologous recombination at neutral site 1 (Clerico et al., Meth. Mol. Biol., (2007) 362:155-71) in the S. elongatus UTEX 2973 genome.

Genetic transformation of S. elongatus: transformation with dsRNA expression vectors was performed as in Vasudevan et al, supra with some modifications. Briefly, tri-parental mating was carried out using microalgae cells, E. coli DH5 alpha cells harboring the dsRNA vector, and E. coli DH5 alpha harboring mobilization plasmid pRL443 (www.addgene.org/70261). Transformants were selected on BG11 medium containing 50 mg/L kanamycin. Transformants were verified by PCR and verified cultures were maintained on agar plates with 50 mg/L kanamycin. For stable transformation, tri-parental mating was carried out as above using vector pPMQAK1-T which contained a dsRNA expression cassette, a spectinomycin resistance cassette, and flanking sequences for homologous recombination at neutral site 1. Transformants were selected on BG11 medium containing 50 mg/L spectinomycin. Segregation of the cargo plasmid was achieved by repeated subcultures of individual colonies in nonselective BG11 liquid media. Individual colonies were screened by PCR for integration at neutral site 1 and for segregation of the cargo plasmid. Verified cultures were maintained on nonselective BG11 agar and checked monthly for stable dsRNA expression. To confirm dsRNA expression by quantitative PCR, approximately 250 mg of fresh biomass was harvested by centrifugation from 7 independent transformant liquid cultures per construct. Two RNA samples were generated from each culture. The first sample consisted of 1 μg DNase-treated total RNA that was used for cDNA synthesis and the second sample consisted of 1 μg DNase-treated total RNA that was treated with dsRNase and used for cDNA synthesis. QPCR was performed using construct-specific primers and the Synechococcus 16S rRNA gene was used for normalization.

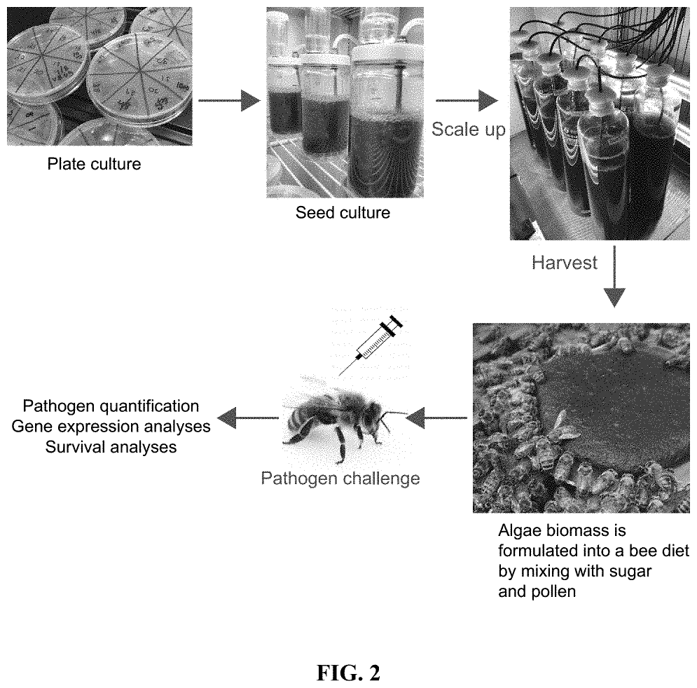

Preparation of engineered microalgae diets for honey bees: Single colonies of transformed cells were grown in 100 ml Erlenmeyer flasks containing 30 ml of liquid BG11 medium and 50 mg/L kanamycin. After 5 days, flask cultures were used to seed 250 ml bottle cultures. Similarly, bottle culture flasks were used to seed larger bottle cultures to obtain 3000 ml primary cultures that were used to generate biomass for feeding experiments (FIG. 2). Cells were harvested via centrifugation and mixed with 50% sucrose syrup (w/v) and 50% pollen (w/w) to formulate the bee diets.

Honey bee feeding setup and experimental design: Experiments were conducted at the USDA-ARS honey bee lab in Baton Rouge, La. Newly emerged worker bees (<24 h old) were obtained by incubating sealed brood combs sourced from healthy colonies at 35° C. and 50% relative humidity (RH). Source colonies were screened via PCR for naturally low virus titers prior to their use in experiments. Bees were collected into a container then randomly assigned to diet treatment cages. First, an experiment was conducted to test the efficacy of dsRNA-expressing microalgae strains to elicit an RNAi response in the bee by knocking down an essential honey bee gene (vitellogenin). Second, experiments were conducted to test the efficacy of dsRNA-expressing microalgae strains to elicit an RNAi response that confers resistance to the RNA virus Deformed Wing Virus. Finally, we tested the potential of microalgae strains targeting Deformed Wing Virus to confer non-specific resistance to the RNA virus Chronic Bee Paralysis Virus.

Knockdown of an endogenous honey bee transcript: We tested the efficacy of dsRNA-expressing microalgae strains to elicit and RNAi response in the bee by knocking down an endogenous gene transcript (vitellogenin). Newly emerged worker bees were fed microalgae diets in cages for 6 days. The different diets consisted of either a YFP dsRNA-expressing strain (non-specific dsRNA control; SEQ ID NO: 1), or two vitellogenin dsRNA-expressing strains that target two different transcript regions—“VgKd1” (SEQ ID NO: 2) and “VgKd2” (SEQ ID NO:3). After 6 days of feeding, a subset of bees was sampled to confirm transcript knockdown via qPCR. Because the vitellogenin gene is involved in bee longevity, we monitored bee survival to confirm that transcript knockdown led to increased mortality in bees that consumed the vitellogenin dsRNA microalgae diets.

Reduction of viral replication and increased survival in experimentally infected honey bees: We performed two experiments testing the efficacy of dsRNA-expressing strains to elicit an RNAi response that confers resistance to the RNA virus Deformed Wing Virus (DWV). In the first experiment, newly emerged worker bees were fed microalgae diets for 4 days. The different diets were comprised of either the wild-type strain (untransformed control), or microalgae strains harboring self-replicating plasmids with the dsRNA expression constructs. The transformed strains included a YFP dsRNA-expressing strain (non-specific dsRNA control), or three DWV dsRNA-expressing strains that target different loci within the viral genome. To obtain viral inoculum for injections, a group of 10 adult bees with phenotypic DWV traits were frozen at −80° C., ground to a fine powder, homogenized in 10 mL sterile 1X PBS, and centrifuged. The supernatants containing the viruses were manually filtered through a 0.2-micron filter to remove small debris, fungi, and bacteria. qPCR was conducted to test for the presence of non-target viruses. Viral quantification for the major DWV variants (DWV-A, DWV-B) was performed by absolute quantification using the Standard Curve Method. To facilitate virus injections, bees were placed in scintillation vials on ice for 2 min to slow movements. Virus treatment was injected at a dose of 10⁶ viral genome equivalents using a 30 G needle inserted into the lateral abdomen between the fourth and fifth pleurites, based on established protocols. An UltraMicroPump with a SYS-Micro4 Controller with an infusion flow rate of 0.1 μl/sec was used, following manufacturer's parameters. 72-hours post injection, bees were collected and assayed for viral titers via qPCR and absolute quantification using a standard curve of DWV plasmid. A second experiment was performed as above with some modifications. Briefly, new microalgae strains were generated in which the dsRNA expression constructs were stably incorporated into the microalgal genome as a single copy. Bees were fed diets comprised of either a vector control strain (no dsRNA expression), a YFP dsRNA-expressing strain (non-specific dsRNA control), or three DWV dsRNA-expressing strains. In this trial, an infectious clone of Deformed Wing Virus (DWV-A) was prepared as previously described (Ryabov et al, PLoS Biol., (2019) 17:e3000502) and used as the source of viral inoculum.

Viral cross-resistance in experimentally infected honey bees: we tested whether DWV dsRNA-expressing microalgae strains can confer resistance to bees experimentally infected with Chronic Bee Paralysis Virus (CBPV). Honey bees exhibiting advanced paralytic symptoms of CBPV were collected from a failing colony and infection was confirmed by PCR using CBPV-specific primers. Individuals were homogenized in sterile PBS and the lysate was clarified by centrifugation and then sterile filtered. The filtrate was diluted by a factor of 10⁻⁶ and 3 μL were injected into white eyed pupae. At 72 hours post injection pupae were snap frozen. The day before intended inoculation the pupae were homogenized in sterile PBS and the homogenate was again prepared by centrifugation and sterilized by filtration. An aliquot was collected for CBPV quantification by quantitative PCR. The inoculum was diluted to 10⁵ CBPV genome equivalents/μL and sucrose was added to a final concentration of 1% to increase viscosity. The inoculum was stored at 4° C. and then used the next day for inoculation. Cages of 55 bees were fed diets comprised of different engineered microalgae strains (vector control, YFP, or DWV1 and DWV2) for 4 days. On the 5^{th} day, cages were CO₂ anesthetized and 500 μL of the inoculum was dispensed into the cage to topically inoculate the entire cage at the same time. The cage was gently agitated until all individuals appeared evenly coated in the inoculum and then cages were placed in a 31° C. incubator and survival was monitored daily.

Results:

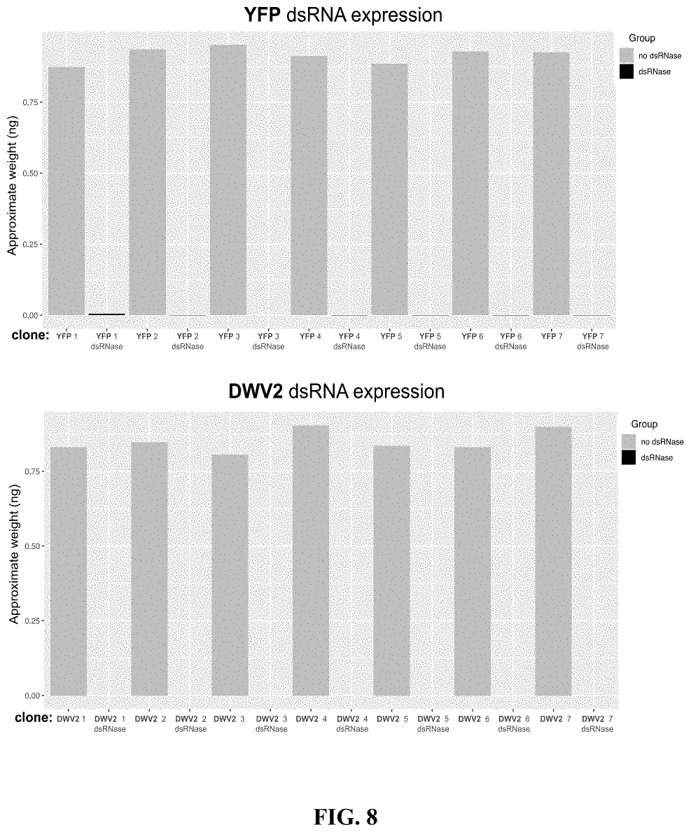

Quantitation of dsRNA production in engineered strains of the prokaryotic microalga Synechococcus elongatus. Seven independent transformants from each of two different dsRNA constructs (top and bottom panels) were screened by quantitative PCR using plasmid standard curves (FIG. 8). Total RNA extracts were either tested directly or treated with RNaseIII, which specifically degrades dsRNA. Fractions of the RNA extracts that were treated with RNaseIII yielded minimal to no amplification, indicating that amplification of untreated extracts is attributable to dsRNA production by the engineered strains.

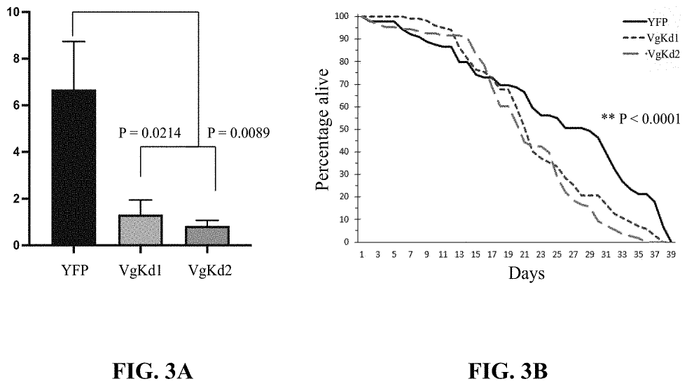

Consumption of dsRNA-expressing microalgae can elicit an RNAi response and knockdown an endogenous honey bee gene. We selected vitellogenin, an essential gene involved in honey bee lifespan regulation, to test gene knockdown efficiency. Bees that consumed microalgae diets comprised of the VgKd1 (SEQ ID NO: 2) and VgKd2 (SEQ ID NO: 3) strains exhibited a significant reduction in vitellogenin mRNA, which consequently led to reduced lifespan (FIG. 3).

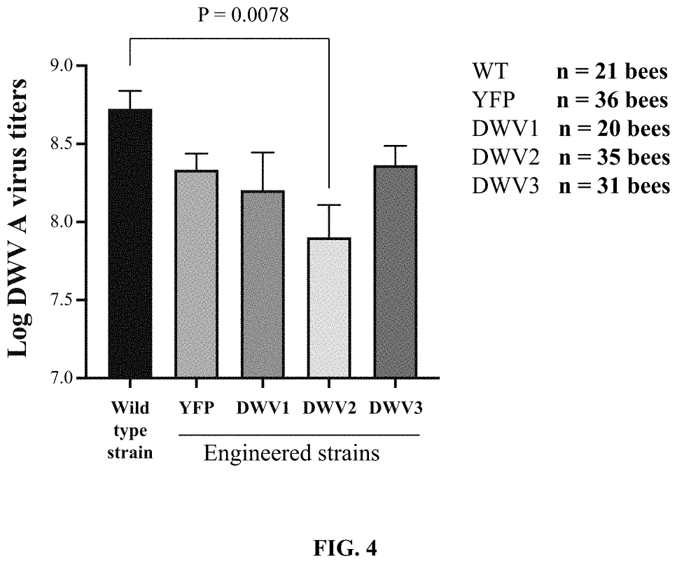

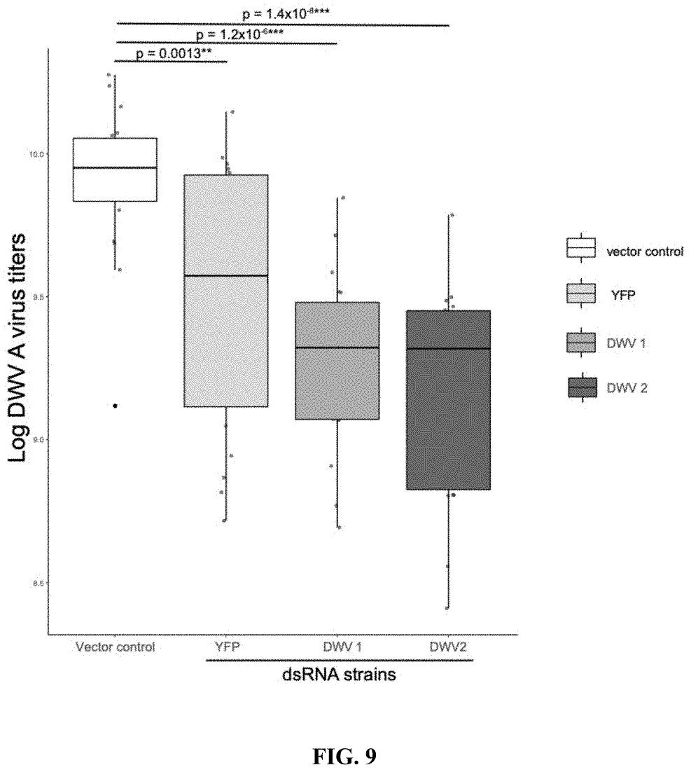

Consumption of Deformed Wing Virus dsRNA-expressing microalgae led to reduced viral titers in experimentally infected honey bees. This result was replicated in two independent tests. Overall, bees that consumed microalgae diets comprised of dsRNA-expressing strains had lower viral titers compared to bees that consumed the wild type algae strain (FIG. 4) and (FIG. 9). In the first test, we observed a statistically significant reduction in viral titers in bees that consumed the DWV2 strain (expressing a dsRNA having the sequence of SEQ ID NO: 5), indicating that this target is more efficacious than the other loci (DWV1 and DWV3, expressing a dsRNA having the sequence of SEQ ID NO: 4 and SEQ ID NO: 6, respectively)). In the second test, microalgae strains that were stably transformed with the dsRNA expression constructs produced a statistically significant reduction of virus titers in bees that consumed the DWV1 and DWV2 strains (FIG. 9).

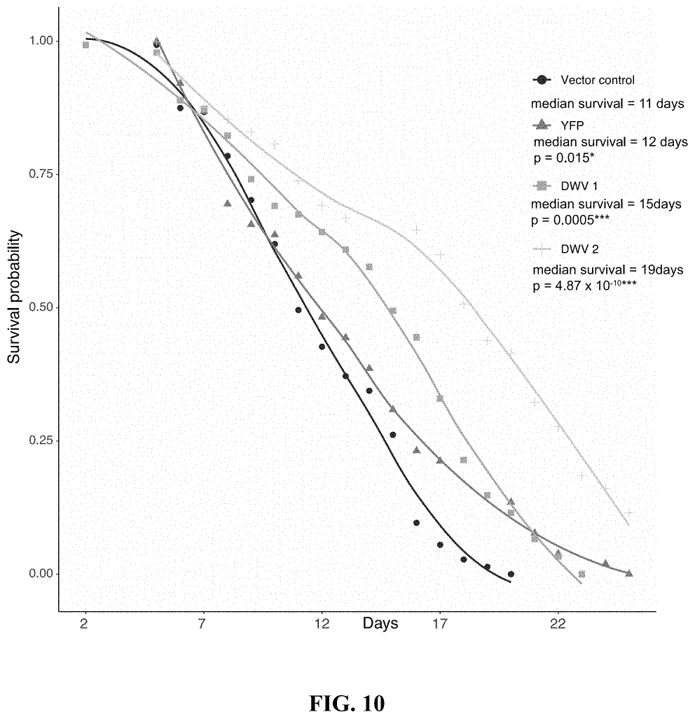

Consumption of Deformed Wing Virus dsRNA-expressing microalgae improves survival of bees experimentally infected with Deformed Wing Virus (DWV). Bees were fed diets formulated with either a vector control strain (no dsRNA expression), a YFP-expressing strain (non-specific dsRNA control), or two microalgae strains expressing fragments DWV1 and DWV2 for 4 days. Bees were then injected with DWV and post injection survival was recorded daily. In agreement with reduced viral titers, experimentally infected bees that fed on the DWV1 and DWV2 strains had significantly improved survival outcomes (FIG. 10).

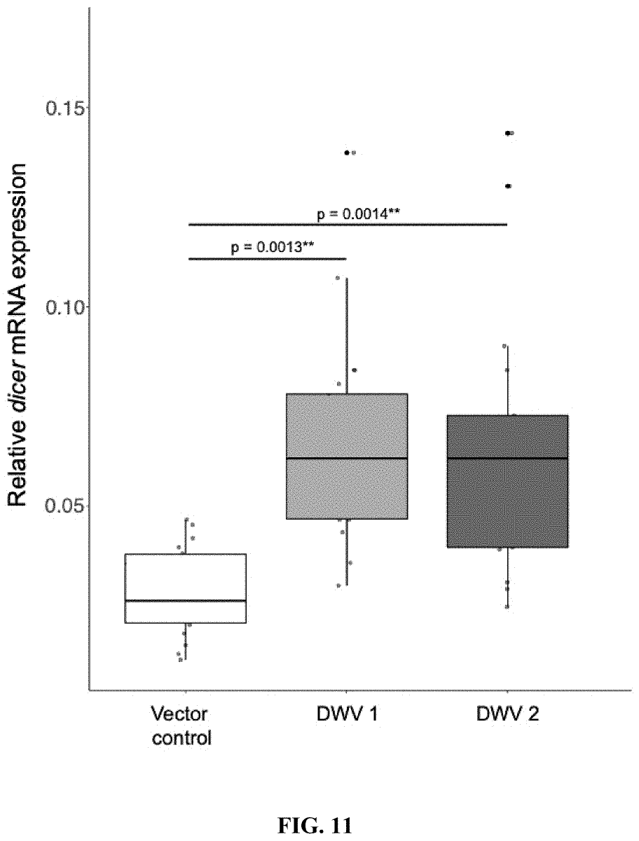

Engineered microalgae diets activate the honey bee RNA interference (RNAi) immune response. Bees were fed diets comprised of either a vector control strain (no dsRNA expression) or two algae strains expressing fragments DWV1 and DWV2 for 4 days. Bees were sampled on day 5 and analyzed for dicer mRNA expression via qPCR. Dicer induces activation of the RNA-induced silencing complex, which is essential for RNAi. Bees fed the DWV1 and DWV2 strains had significantly higher dicer expression relative to bees fed the vector control diet (FIG. 11).

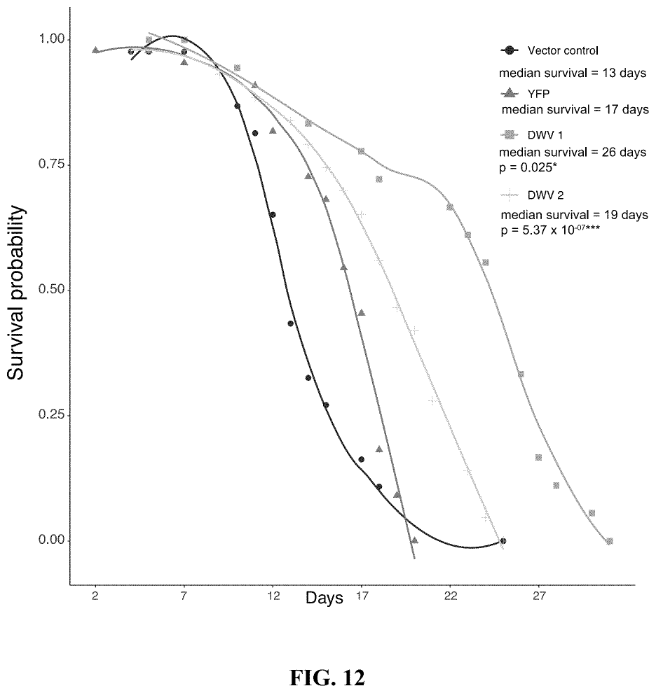

Engineered microalgae diets expressing fragments DWV1 and DWV2 confer viral cross-resistance to bees experimentally infected with Chronic Bee Paralysis Virus (CBPV). Bees were fed diets formulated with either a vector control strain (no dsRNA expression), a YFP-expressing strain (non-specific dsRNA control), or two microalgae strains expressing fragments DWV1 and DWV2 for 4 days. Bees were topically infected with CBPV and post infection survival was recorded daily. Bees that fed on the DWV1 and DWV2 strains had significantly improved survival outcomes (FIG. 12), indicating that these strains can confer resistance to CBPV.

### Example 2

Evaluation of Nutritional Efficacy of Other Microalgae

We evaluated the nutritional efficacy of the prokaryotic alga Athrospira platensis (commonly called “spirulina”) and the eukaryotic alga, Chlorella vulgaris to test their relative nutritional value to honey bees when compared to pollen, the bees natural diet.

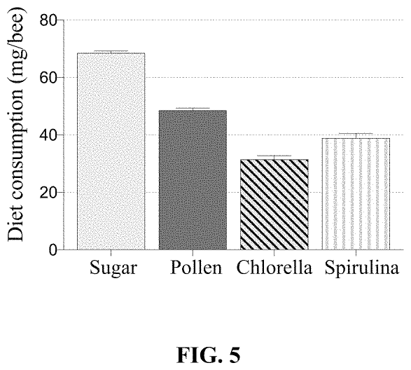

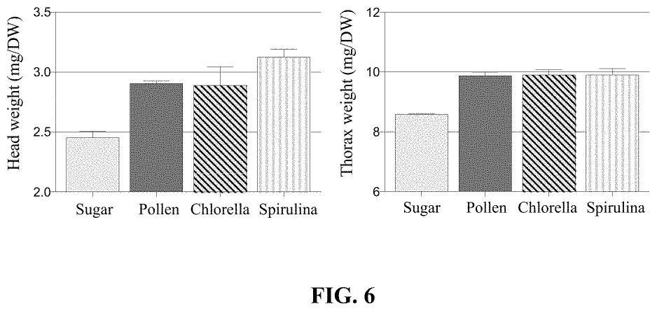

Newly emerged, caged honey bees were fed diets of either sucrose (negative control), pollen (positive control), dried A. platensis (spirulina) and dried C. vulgaris (Chlorella). The following parameters were measured after 7 days of ad libitum feeding: diet consumption, bodyweight gain, nutritional gene expression, and stress response gene expression. Bees consumed significantly less of the microalgae diets (FIG. 5) yet had similar bodyweights (head and thorax) relative to a natural pollen diet (FIG. 6). This finding indicates that microalgae nutrition is highly bioavailable to honey bees.

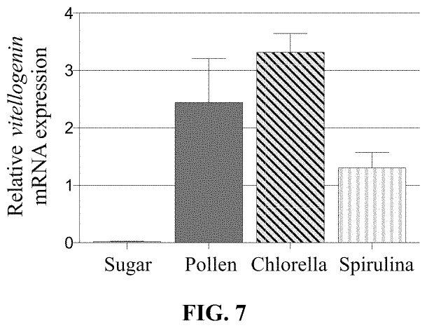

Gene expression analyses were conducted to assess the impact of the different diets on nutrition-related gene expression. Transcript levels were measured using standard qPCR protocols. Honey bee vitellogenin is a critically important, nutritional storage protein that is primarily regulated at the transcriptional level by diet quality and quantity. We found that the Chlorella diet led to significantly elevated vitellogenin mRNA levels that trended towards higher levels than a natural pollen diet, despite significantly reduced consumption of the Chlorella diet (FIG. 7).

Although transcript levels were on average 63-fold higher than the sugar negative control diet, bees fed spirulina had significantly lower vitellogenin levels than pollen- and Chlorella-fed bees. Dietary lipid content plays a regulatory role in vitellogenin expression (Wegener et al., Insectes Sociaux, (2018) 65:381-91) and prokaryotic microalgae generally accumulate fewer lipids than eukaryotic microalgae. This indicates potential to augment prokaryotic microalgae diets via genetic engineering or by mixing them with Chlorella or other eukaryotic algae biomass that accumulates higher lipid concentrations.

### Example 3

Enhanced Nutrition

Vitellogenin (Vg) is the main nutritional storage protein in honey bees and is the precursor to proteinacious jelly secretions that are produced by worker bees to feed young larvae. We will generate an expression construct for production of recombinant honey bee Vg protein in S. elongatus 2973. Honey bee Vg has a high content of leucine, lysine, valine, threonine, and isoleucine, amino acids which are essential to honey bees. Strains are developed that co-express dsRNAs and recombinant Vg. This modified microalga is tested for improvements in the nutritional value of dsRNA-producing microalgae used as bee feed. The gene sequence for honey bee Vg will be codon-optimized (SEQ ID NO: 7) for expression in other prokaryotic microalgae species.

Methods: Honey bee (Apis mellifera) Vg sequence is obtained from Genbank (accession #NM_001011578.1). Bases 1-24 are truncated from the sequence to remove a signal peptide. The gene sequence is then codon optimized in silico for expression in S. elongatus 2973 and synthesized by Integrated DNA Technologies. The synthetic gene is placed under the control of the constitutive Ptrc10 promoter and TrrnB terminator. A construct similar to those described above for expression of dsRNA in microalgae will also contain the synthetic Vitellogenin. The microalga transformed with this construct will be tested as a bee feed component.

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A recombinant prokaryotic microalga, the recombinant prokaryotic microalga comprising a heterologous nucleic acid encoding a double-stranded ribonucleic acid (dsRNA), wherein the dsRNA targets a gene expressed by a bee or a gene expressed by a bee pathogen and wherein the dsRNA results in knockdown or silencing of the dsRNA gene target within the bee.

2. The microalga of claim 1, wherein the bee is a honey bee.

3. The microalga of claim 1, wherein the bee is a domesticated bee of the species Apis mellifera, Apis cerana, or Bombus impatiens.

4. The microalga of claim 1, wherein the microalga is Synechococcus elongatus, Arthrospira platensis, or Arthrospira maxima.

5. The microalga of claim 4, wherein the microalga is Synechococcus elongatus.

6. The micro alga of claim 1, wherein the bee pathogen is a honey bee pathogen.

7. The microalga of claim 6, wherein the honey bee pathogen is a virus.

8. The microalga of claim 7, wherein the virus is Deformed Wing Virus (DWV).

9. The microalga of claim 1, wherein the microalga comprises a lipid that is an essential nutrient for the bee.

10. A bee feed comprising the recombinant microalga of claim 1.

11. A method of inducing RNA interference (RNAi) in a bee or bee pathogen, comprising the steps of: 1) contacting an artificial bee feed comprising the recombinant prokaryotic microalga of claim 1 with a bee; and 2) allowing the bee to ingest the microalga, thereby ingesting an effective amount of dsRNA produced by said microalga to induce RNAi.

12. The microalga of claim 11, wherein the bee is a honey bee.

13. The microalga of claim 11, wherein the bee is a domesticated bee of the species Apis mellifera, Apis cerana, or Bombus impatiens.

14. The microalga of claim 11, wherein the microalga is Synechococcus elongatus, Arthrospira platensis, or Arthrospira maxima.

15. The microalga of claim 14, wherein the microalga is Synechococcus elongatus.

16. A double-stranded RNA (dsRNA), comprising a nucleic acid having the sequence of SEQ ID NO: 4.
